(19) United States
(12) Patent Application Publication
(10) Pub. No.: US 2022/0257170 A1
(43) Pub. Date: Aug. 18, 2022

(54) STIMULATING SYSTEM FOR COLLABORATIVE FUNCTIONS OF BRAIN AND BODY

(71) Applicant: CHULALONGKORN UNIVERSITY, Bangkok (TH)

(72) Inventors: Solaphat HEMRUNGROJN, Bangkok (TH); Nutnicha PENSRESIRIKUL, Bangkok (TH)

(21) Appl. No.: 17/734,189

(22) Filed: May 2, 2022

Related U.S. Application Data
(63) Continuation of application No. PCT/TH2020/000074, filed on Oct. 29, 2020.

(30) Foreign Application Priority Data
Oct. 30, 2019 (TH) 1901006890

(51) Int. Cl. A61B 5/00

(52) U.S. Cl. CPC A61B 5/4064; A61B 5/6892; A61B 5/6814; A61B 5/743; A61B 5/6887; A61B 5/741

(57) ABSTRACT

A stimulating system for collaborative functions of brain and body according to this invention comprising a touch sensor which senses a user's input and transmits the detected input signals when at least one part of user's body touches at least one predefined area of the said sensor, a stimulating pattern generator which contains instruction directing a user to touch the sensor in a predefined pattern, a stimulating pattern display unit which displays the instruction corresponding to the said stimulating pattern for the user, a synergistic operation processor which processes the efficiency in the synergistic operations of brain and body and the stimulating pattern generator wherein the said synergistic operation processor is configured to receive touch data from the said touch sensor, examine a relationship between the said data and the pattern defined by the stimulating pattern generator and process such relationship in the form of a relationship score based on the predefined levels of relationship.

### FIELD OF INVENTION

This invention is related to a stimulating system for collaborative functions of brain and body

### BACKGROUND

After seeing the problems of the elderly with regard to the body and brain, the research team from the Department of Psychiatry, Faculty of Medicine, Chulalongkorn University together with the research team from Computer Engineering Department, Faculty of Engineering, Chulalongkorn University and the research team from the technological operation laboratory for rehabilitation and facilitation, Biomedical Electronics and Systems Research Unit, National Electronics and Computer Technology Center studied the effects of brain exercises in the elderly via the method of meta-analysis and found that the exercises significantly affect the brain's operation. Therefore, there was a concept to organize research in relation to the systems and methods for promoting skill-related Physical Fitness that support and enable good exercise together with the rehabilitation of the cognitive level for the elderly in order to enhance potential relating to cognition, learning, and short-term memorization as well as the ability to plan and problem solving skills.

From the prior art searches, even though there are works disclosing the stimulation of cognitive levels for elderly, said works are specific and do not cover the application in an integrated manner. Particularly, the prior arts did not disclose the mixed stimulation at various levels which are appropriate for the different stages of old age. Therefore, there is a need to invent a system that can provide appropriate and clear stimulation and application.

### SUMMARY OF THE INVENTION

One of the objectives of this invention is to provide a system and present procedures for the elderly so that the elderly can utilize physical ability in relation to brain operation. With different stimulation methods for enhancing concentration and maintaining such concentration for a certain period of time, the system and methods as disclosed emphasize body and brain training at the same time by improving cognitive ability in terms of the attention, memory, and psychomotor speed, executive function, social emotional cognitive, physical fitness, physical strength, physical balance, and the training of synergistic operations between the brain and body.

A stimulating system for collaborative functions of brain and body according to this invention comprises a touch sensor which senses a user's input and transmits the detected input signals when at least one part of user's body touches at least one predefined area of the said sensor wherein the said sensor stores the user's touching data comprising at least the touching positions and the differences between the touching times and the times that the stimulating patterns are displayed; a stimulating pattern generator which comprises at least a processor generating the stimulating patterns and a memory unit connected to the said processor containing an instruction directing a user to touch the sensor in a predefined pattern; a stimulating pattern display unit which displays the instruction corresponding to the said stimulating pattern for the user wherein the said display unit is connected to the stimulating pattern generator and the touch sensor; a synergistic operation processor which processes the efficiency in the synergistic operations of brain and body and is connected the touch sensor and the stimulating pattern generator wherein the said synergistic operation processor is configured to receive touch data from the said touch sensor and examine a relationship between the said data and the pattern defined by the stimulating pattern generator and process such relationship in the form of a relationship score based on the predefined levels of relationship; an efficiency display unit which is connected to the synergistic operation processor and displays the efficiency in the synergistic operations of brain and body based on the relationship score characterized in that the stimulating pattern generator generates a plurality of images wherein each image comprises at least a set of objects in which each object has a predefined color, size, position, display order, and the relationship of the said characteristics with those of the other objects in the image and designates such group of images as the original pattern and subsequently generates a subsequent pattern having the predefined level and number of differences when compared to the characteristics of the original pattern and the stimulating pattern generator transmits the original pattern and the second pattern to the stimulating pattern display unit and that the said stimulating pattern display unit displays said patterns in the predefined timeframe based on the number of patterns to be displayed and wherein a subsequent pattern generated by the stimulating pattern generator comprises more than one differences which are grouped into the primary difference and secondary difference in which the secondary difference is the difference within the defined area of the primary difference and comprises the predefined scope of different characteristics and number of said differences and wherein the synergistic operation processor processes the relationship by specifying the first weighing value for the relationship between the touch data and the stimulating patterns when there is one visual difference and further specifying the second weighing value for the relationship between touch data and the stimulating patterns when the stimulating patterns comprises the primary difference and secondary difference and further specifying the third weighing value for the predefined difference of the touching times wherein the second weighing value is more than the first weighing value and the third weighing value is less than the first and the second weighing values.

In one aspect, the number of stimulating patterns can be different from the original pattern and comprise the number of patterns having primary and secondary differences in the range of 20-40% of the total number of patterns, and preferably 30% of the total number of patterns. In this regard, the secondary difference is the reversed characteristic of the primary characteristic.

In the other aspect, the stimulating pattern generator changes the defined relationship to be different or opposite to the original predefined relationship.

In the other aspect, the stimulating pattern generator reduces the number of differences of the stimulating patterns subsequently generated when the subsequent efficiency calculated from the synergistic operation processor is lower than the efficiently previously calculated.

Further, the stimulating pattern generator can specify the differences based on the conditional information provided by a user which comprises predefined age information and educational level information.

The stimulating system for collaborative functions of brain and body may also comprise a brain wave detector which is connected to at least one part of body for receiving brain wave signals and a brain wave processor and a memory unit connected to the said brain wave processor wherein the brain wave signals are stored in the memory unit and the brain wave processor processes the said stored brain wave signals based on the predefined range of brainwave signals and transmits the value of the difference between the values based on the detected signal and the predefined signal to the stimulating pattern generator.

In this regard, the predefined range of brainwave signals comprise more than one range and each range corresponds to the level of relationship between the patterns and number of differences between the patterns to be generated by the stimulating pattern generator.

In the other aspect, the synergistic operation processor processes the data by distinguishing the relationship into at least 3 levels. The synergistic operation processor may process the aggregated relationship data based on the predefined number of data to be processed and categorize them into the levels as aforementioned.

The synergistic operation processor may also reduce number of data to be processed when there is no relationship found.

In the other aspect, the image set is a set of related images continuously displayed and the display unit may display the stimulating pattern comprising a set of images for 3 minutes per set.

The display unit further may also comprise an image to audio convertor which converts the visual differences into audio data and generates the corresponding audio signal for a user.

In the other aspect, touch data further comprising the data of the body part touching the predefined area and the corresponding positioning data being set as an initial position or the information on the touching force levels or both positioning data and force level data which are calculated and stored based on the predefined weight for each type of data.

In the other aspect, the touch sensor comprises a plurality of objects arranged to be positioned on the base attached to the ground and each comprises at least one area for sensing user's touch. Such objects can be dome-like objects arranged to be positioned on the ground in a circular manner having the user's standing position as a center.

In the other aspect, the number of dome-like objects is 6 and the distance of the radius between the center of the circle and the positions of dome-like objects is in the range of 30 to 80 cm and the diameter of the dome-like objects is in the range of 8-20 cm and the height from the base is in the range of 15 to 50 cm. However, if the number of dome-like objects is 12, the distance and the radius can be varied.

The objectives and unique characteristics and other aspects of this invention will be described in more detail by way of the examples and drawings included and the best mode will also be further described.

### BRIEF DESCRIPTION OF THE DRAWINGS

FIG. 1 shows the image of the template which is different from the original template having the differences according to example No. 1.

FIG. 2 shows the image of the template which is different from the original template having the differences according to example No. 2.

FIG. 3 shows the image of the template which is different from the original template having the differences according to example No. 3.

FIG. 4 shows the image of the template which is different from the original template having the differences according to example No. 4.

FIG. 5 shows one of the possible embodiments of the touch sensor and the arrangement of the same.

FIG. 6 shows the example of the system according to this invention.

### DETAILED DESCRIPTION OF PREFERRED EMBODIMENTS

For a better understanding of the preferred embodiment and to show how it may be performed, it will now be described in more detail by way of examples only with reference to the accompanying drawings. The parts shown in the drawings will be represented by the referenced numbers. The description, however, does not imply to any limitation, and the scope of the invention will be in accordance with the claims attached herein.

A stimulating system for collaborative functions of brain and body according to this invention comprising a touch sensor which senses a user's input and transmits the detected input signals when at least one part of user's body touches at least one predefined area of the said sensor wherein the said sensor stores the user's touching data comprising at least the touching positions and the differences between the touching times and the times that the stimulating patterns are displayed; a stimulating pattern generator which comprises at least a processor generating the stimulating patterns and a memory unit connected to the said processor containing an instruction directing a user to touch the sensor in a predefined pattern; a stimulating pattern display unit which displays the instruction corresponding to the said stimulating pattern for the user wherein the said display unit is connected to the stimulating pattern generator and the touch sensor; a synergistic operation processor which processes the efficiency in the synergistic operations of brain and body and is connected the touch sensor and the stimulating pattern generator wherein the said synergistic operation processor is configured to receive touch data from the said touch sensor and examine a relationship between the said data and the pattern defined by the stimulating pattern generator and process such relationship in the form of a relationship score based on the predefined levels of relationship; an efficiency display unit which is connected to the synergistic operation processor and displays the efficiency in the synergistic operations of brain and body based on the relationship score characterized in that the stimulating pattern generator generates a plurality of images wherein each image comprises at least a set of objects in which each object has a predefined color, size, position, display order, and the relationship of the said characteristics with those of the other objects in the image and designates such group of images as the original pattern and subsequently generates a subsequent pattern having the predefined level and number of differences when compared to the characteristics of the original pattern and the stimulating pattern generator transmits the original pattern and the second pattern to the stimulating pattern display unit and that the said stimulating pattern display unit displays said patterns in the predefined timeframe based on the number of patterns to be displayed and wherein a subsequent pattern generated by the stimulating pattern generator comprises more than one differences which are grouped into the primary difference and secondary difference in which the secondary difference is the difference within the defined area of the primary difference and comprises the predefined scope of different characteristics and number of said differences and wherein the synergistic operation processor processes the relationship by specifying the first weighing value for the relationship between the touch data and the stimulating patterns when there is one visual difference and further specifying the second weighing value for the relationship between touch data and the stimulating patterns when the stimulating patterns comprises the primary difference and secondary difference and further specifying the third weighing value for the predefined difference of the touching times wherein the second weighing value is more than the first weighing value and the third weighing value is less than the first and the second weighing values.

In one aspect, the number of stimulating patterns can be different from the original pattern comprising the number of patterns having primary and secondary differences in the range of 20-40% of the total number of patterns, and preferably 30% of the total number of patterns.

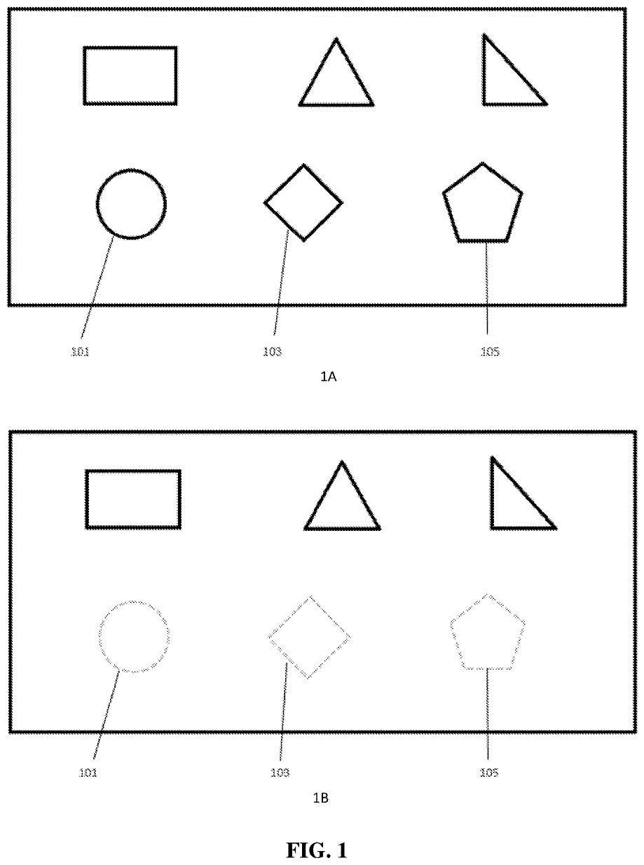

In one of the examples according to this invention, the stimulating pattern generator generates a visual pattern different from the original visual pattern as shown in FIG. 1. The display order and the colors of a certain number of visual objects are also different from those of the original pattern. As shown in FIG. 1a, the first object (101) is displayed first and the second object (103) and the third object (105) are subsequently displayed in order. Each object has a different color. After the display, as shown in FIG. 1b, the said object is configured to be changed. In this example, the object is configured to be varnished. The instructions given to the user in this case are to touch the touch sensor in the manner that corresponds to the order of display and the color of the visual object in the stimulating pattern which relates to the original visual pattern.

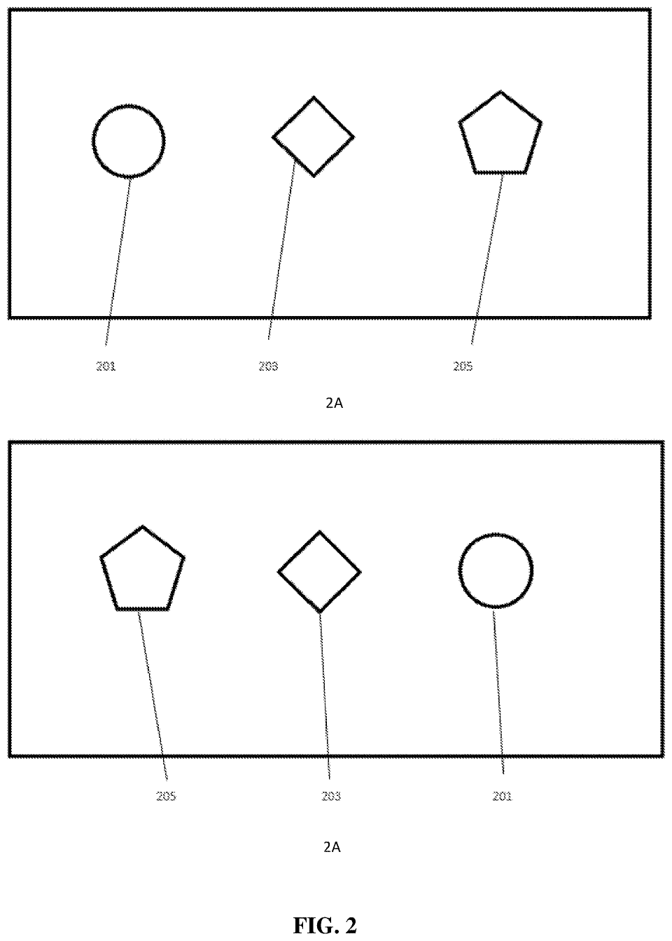

The change can be configured in more than one manner. The other possible alternative is as shown in FIG. 2a in which the first object (201) is displayed first. The second object (103) and the third object (105) are subsequently displayed in order. Each object has a different color. After the display, as shown in FIG. 1b, the said object is configured to be changed. As shown in FIG. 2b, the third object (205) is displayed first this time. The second object (203) and the first object (201) are subsequently displayed in order. The instructions given to the user in this case is to touch the touch sensor in the manner that corresponds to the order of display and the color of visual object in the stimulating pattern which relates to the original visual pattern.

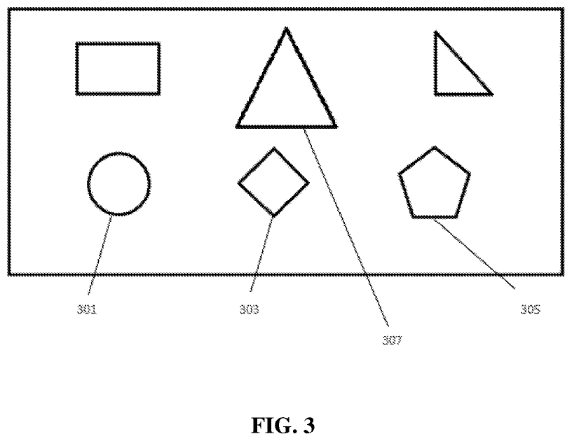

One of the unique characteristics of this invention is to change the characteristics of an object. This can include the changes in terms of the increase or decrease of the number and size of an object. In case of the increase, the objects in example 1 and 2 above can be additionally changed in which the direction of change can correspond or can be opposite to the display order and colors of the objects in the original pattern. As shown in FIG. 3, the objects are displayed in order namely the first object (301), the second object (303), and the third object (305). However, the fourth object (307) which is not configured in the original template appears larger to gain the attention of the user.

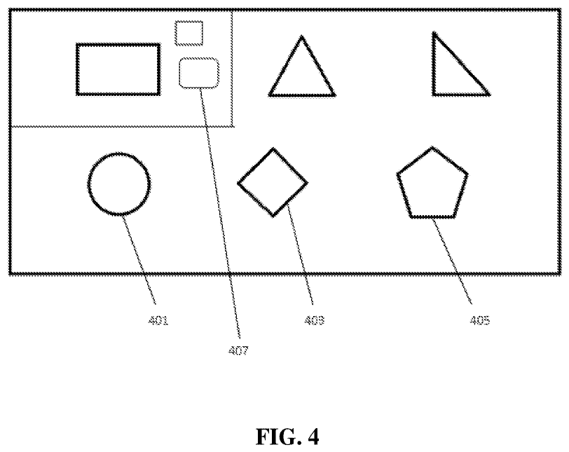

In the other possible alternatives regarding the change, such alternatives may cover changes of the property and number of objects. As shown in FIG. 4, the sub-area of the visual image provided comprises a plurality of objects which are configured to be changed in the manner corresponding to the visual object in the original template and the additional object has certain properties relating to the objects configured to be changed. These properties are intended to attract the user's attention. In the example according to FIG. 4, the objects to be displayed in order are the first object (401), the second object (403), and the third object (405). However, in the sub-are around the first object, there is an additional object (407) having characteristics different from those of the first object such as there are other colors used in the text captions. This is different from the first object having a display of the shades of colors.

The stimulating pattern generator may reduce the number of differences in the stimulating patterns subsequently generated when the subsequent efficiency calculated from the synergistic operation processor is lower than the efficiently previously calculated. The stimulating pattern generator may also specify the differences based on the conditional information provided by a user which comprises predefined age information and educational level information.

The stimulating system for collaborative functions of brain and body can further comprise a brain wave detector which is connected to at least one part of body for receiving brain wave signals and a brain wave processor and a memory unit connected to the said brain wave processor. The brain wave signals are stored in the memory unit and the brain wave processor processes the said stored brain wave signals based on the predefined range of brainwave signals and transmits the value of the difference between the values based on the detected signal and the predefined signal to the stimulating pattern generator. When the stimulating pattern generator receives the signal, the said stimulating pattern generator will generate a stimulating pattern different from the original pattern. The number of differences may be lower when the brain wave signal is lower than the predefined threshold and higher when the signal is higher than the threshold. The optimal number of different levels may be 3.

For the display, the display unit may display the stimulating pattern in the form of related image sets continuously and in animated fashion and may set the display time to be 3 minutes per set.

In the other aspect, the stimulating pattern generator may additionally set the condition in order to change the defined relationship between the stimulating pattern and the original pattern during the use so that certain characteristic of the stimulating pattern will be different or opposite to those of the original predefined relationship. In such case, the display unit will display the instructions based on the changed relationship to the user.

In the other aspect, the display unit may further comprise an image to audio convertor which converts the visual differences into audio data and generates the corresponding audio signal for a user. In such case, the user will receive the instructions corresponding to the visual image in the audio format.

A synergistic operation processor which processes the efficiency in the synergistic operations of the brain and body is configured to connect with the touch sensor and the stimulating pattern generator. In this regard, the said synergistic operation processor is configured to receive touch data from the said touch sensor, examine a relationship between the said data and the pattern defined by the stimulating pattern generator, and process such relationship in the form of a relationship score based on the predefined levels of relationship.

The synergistic operation processor processes the relationship by specifying the first additional weighing value for the relationship between touch data and the stimulating patterns when the stimulating patterns comprises the primary difference and secondary difference and further specifying the second additional weighing value when the difference of the touching times are within the predefined range. The synergistic operation processor processes the relationship data by taking the weighting values into account. When there are more than one type of data differences and there is a time difference regarding the touching, the synergistic operation processor will process the relationship as aggregated data again and such aggregated data will be categorized according to the predefined ranges and interpreted as the level of the synergistic operation of the brain and body.

In one aspect, the synergistic operation processor will process the relationship as aggregated data based on the predefined levels of relationship. In this example, there are 4 levels, namely, above average cognitive disorder, low level cognitive disorder, normal level, and a very good level of cognition.

In the other aspect, the measurement of efficiency can be conducted based on the statistical information including the percentiles from the group of users which can also be regarded as the criteria for data categorization. When the values representing the aforementioned relationship and assigned weights lead to high accuracy, the corresponding range of percentile may be adopted for determining as to whether the efficiency is high. However, the various ranges of percentile may also be used and each range may not be equal. For examples, the ranges of percentile can be 1-25, 26-50, 51-70, and 71-100.

In the other aspect, touch data further comprises the data of the body part touching the predefined area and the corresponding positioning data which is regarded as an initial position or the information on the touching force levels or both positioning data and force level data which are calculated and stored based on the predefined weight for each type of data.

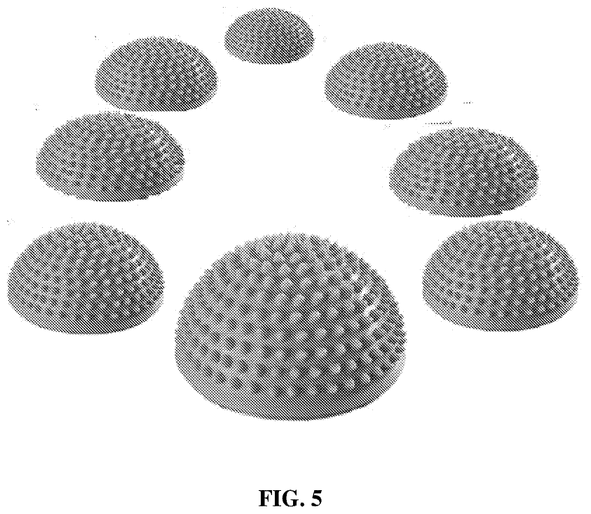

As shown in FIG. 5, the touch sensor comprises a plurality of objects arranged to be positioned on the base attached to the ground and each comprises at least one area for sensing the user's touch. Such objects can be multiple dome-like objects arranged to be positioned on the ground in the circular manner having the user's standing position as a center.

In case that the number of dome-like objects is 6, the distance of the radius between the center of the circle and the positions of dome-like objects is in the range of 30 to 80 cm and the diameter of the dome-like objects is in the range of 8-20 cm and the height from the base is in the range of 15 to 50 cm. However, if the number of dome-like objects is 12, the distance and the radius can be varied.

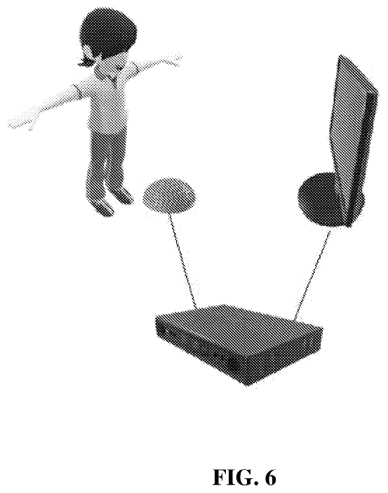

The example of the system as a whole is shown in FIG. 6 in which the user can touch the touch sensor which is connected to the stimulating pattern generator and the synergistic operation processor, the stimulating pattern display unit, the efficiency display unit. Such components can be arranged to be within one housing and the display units are both configured to connect to the external displays.

It will be appreciated by persons skilled in the art that the present inventions are not limited by what has been particularly shown and described herein above. Rather the scope of the present invention includes both combinations and sub-combinations of the features described herein above as well as modifications and variations thereof which would occur to a person of skill in the art upon reading the foregoing description and which are not in the prior art.

### BEST MODE

As disclosed in the Details of the Preferred Embodiments.

1. A stimulating system for collaborative functions of brain and body comprising:
a touch sensor which senses a user's input and transmits the detected input signals when at least one part of user's body touches at least one predefined area of the said sensor wherein the said sensor stores the user's touching data comprising at least the touching positions and the differences between the touching times and the times that the stimulating patterns are displayed;
a stimulating pattern generator which comprises at least a processor generating the stimulating patterns and a memory unit connected to the said processor containing an instruction directing a user to touch the sensor in a predefined pattern;
a stimulating pattern display unit which displays the instructions corresponding to the said stimulating pattern for the user wherein the said display unit is connected to the stimulating pattern generator and the touch sensor;
a synergistic operation processor which processes the efficiency in the synergistic operations of the brain and body and is connected the touch sensor and the stimulating pattern generator wherein the said synergistic operation processor is configured to receive touch data from the said touch sensor, examine a relationship between the said data and the pattern defined by the stimulating pattern generator, and process such relationship in the form of a relationship score based on the predefined levels of relationship;
an efficiency display unit which is connected to the synergistic operation processor and displays the efficiency in the synergistic operations of the brain and body based on the relationship score characterized in that the stimulating pattern generator generates a plurality of images wherein each image comprises at least a set of objects in which each object has a predefined color, size, position, display order, and the relationship of the said characteristics with those of the other objects in the image and designates such group of images as the original pattern and subsequently generates a subsequent pattern having the predefined level and number of differences when compared to the characteristics of the original pattern and the stimulating pattern generator transmits the original pattern and the second pattern to the stimulating pattern display unit and that the said stimulating pattern display unit displays said patterns in the predefined timeframe based on the number of patterns to be displayed,
wherein a subsequent pattern generated by the stimulating pattern generator comprises more than one difference which are grouped into the primary difference and secondary difference in which the secondary difference is the difference within the defined area of the primary difference and comprises the predefined scope of different characteristics and number of said differences,
wherein the synergistic operation processor processes the relationship by specifying the first weighing value for the relationship between the touch data and the stimulating patterns when there is one visual difference and further specifying the second weighing value for the relationship between the touch data and the stimulating patterns when the stimulating patterns comprises the primary difference and secondary difference and further specifying the third weighing value for the predefined difference of the touching times, and
wherein the second weighing value is more than the first weighing value and the third weighing value is less than the first and the second weighing values.

2. A stimulating system for collaborative functions of brain and body as cited in claim 1, wherein the number of the stimulating patterns which are different from the original pattern comprises the number of patterns having primary and secondary differences in the range of 20-40% of the total number of patterns, and preferably 30% of the total number of patterns.

3. A stimulating system for collaborative functions of brain and body as cited in claim 1, wherein the secondary difference is the reversed characteristic of a primary characteristic.

4. A stimulating system for collaborative functions of brain and body as cited in claim 1, wherein the stimulating pattern generator changes the defined relationship to be different or opposite to the original predefined relationship.

5. A stimulating system for collaborative functions of brain and body as cited in claim 1, wherein the stimulating pattern generator reduces the number of differences of the stimulating patterns subsequently generated when the subsequent efficiency calculated from the synergistic operation processor is lower than the efficiently previously calculated.

6. A stimulating system for collaborative functions of brain and body as cited in claim 1, wherein the stimulating pattern generator specifies the differences based on the conditional information provided by a user which comprises predefined age information and educational level information.

7. A stimulating system for collaborative functions of brain and body as cited in claim 1, further comprising a brain wave detector which is connected to at least one part of body for receiving brain wave signals and a brain wave processor and a memory unit connected to the said brain wave processor wherein the brain wave signals are stored in the memory unit and the brain wave processor processes the said stored brain wave signals based on the predefined range of brainwave signals and transmits the value of the difference between the values based on the detected signal and the predefined signal to the stimulating pattern generator.

8. A stimulating system for collaborative functions of brain and body as cited in claim 7, wherein the predefined range of brainwave signals comprise more than one range and each range corresponds to the level of relationship between the patterns and number of differences between the patterns to be generated by the stimulating pattern generator.

9. A stimulating system for collaborative functions of brain and body as cited in claim 1, wherein the synergistic operation processor processes the data by distinguishing relationships into at least 3 levels.

10. A stimulating system for collaborative functions of brain and body as cited in claim 1, wherein the synergistic operation processor processes the aggregated relationship data based on the predefined number of data to be processed.

11. A stimulating system for collaborative functions of brain and body as cited in claim 8, wherein the synergistic operation processor reduces number of data to be processed when there is no relationship found.

12. A stimulating system for collaborative functions of brain and body as cited in claim 1, wherein the image set is a set of related images continuously displayed.

13. A stimulating system for collaborative functions of brain and body as cited in claim 1, wherein the display unit displays the stimulating pattern comprising a set of images for 3 minutes per set.

14. A stimulating system for collaborative functions of brain and body as cited in claim 1, wherein the display unit further comprising an image to audio convertor which converts the visual differences into audio data and generates the corresponding audio signal for a user.

15. A stimulating system for collaborative functions of brain and body as cited in claim 1, wherein touch data further comprising the data of the body part touching the predefined area and the corresponding positioning data being set as an initial position or the information on the touching force levels or both positioning data and force level data which are calculated and stored based on the predefined weight for each type of data.

16. A stimulating system for collaborative functions of brain and body as cited in claim 1, wherein the touch sensor comprises a plurality of objects arranged to be positioned on the base attached to the ground and each comprises at least one area of sensing user's touch.

17. A stimulating system for collaborative functions of brain and body as cited in claim 16, wherein the touch sensor comprises a plurality of dome-like objects arranged to be positioned on the ground in the circular manner having the user's standing position as a center.

18. A stimulating system for collaborative functions of brain and body as cited in claim 17, wherein the number of dome-like objects is 6 and the distance of the radius between the center of the circle and the positions of dome-like objects is the range of 30 to 80 cm and the diameter of the dome-like objects is in the range of 8-20 cm and the height from the base is in the range of 15 to 50 cm.

19. A stimulating system for collaborative functions of brain and body as cited in claim 17, wherein the number of dome-like objects is 12.
